(19) United States
(12) Patent Application Publication
(10) Pub. No.: US 2006/0020043 A1
(43) Pub. Date: Jan. 26, 2006

(54) Methods and compositions for reducing C-reactive protein

(76) Inventor: Roger Berlin, Mendham, NJ (US)

Correspondence Address: WYETH;PATENT LAW GROUP, 5 GIRALDA FARMS, MADISON, NJ, 07940, US

(21) Appl. No.: 10/898,886

(22) Filed: Jul. 26, 2004

(51) Int. Cl. A61K 31/045

(52) U.S. Cl. 514/724

(57) ABSTRACT

The present invention provides methods for the treatment and/or amelioration of inflammation in a subject, including humans, by administering to a subject a therapeutically effective amount of a biologically active mixture of high purity, high molecular weight straight chain primary aliphatic alcohols (referred to collectively herein as policosanol). Also included are methods of reducing levels of C-reactive protein in a subject, comprising administering to a subject a therapeutically effective amount of policosanol.

### BACKGROUND OF THE INVENTION

1. Field of the Invention

The present invention relates to therapeutic compositions and methods for the treatment and/or amelioration of inflammation in a subject, including humans, by administering to a subject a therapeutically effective amount of a biologically active mixture of high purity, high molecular weight straight chain primary aliphatic alcohols (referred to collectively herein as policosanol). Also included are methods of reducing levels of C-reactive protein in a subject, comprising administering to a subject a therapeutically effective amount of policosanol.

2. Description of the State of Art

Inflammation and associated inflammatory responses are important components of host protection to a variety of insults, which may be infectious or non-infectious in nature. While specific responses to an injury or insult may vary, the “inflammatory response” can be viewed as a composite response including successive events in response to a stimulus. Thus, inflammation involves a number of cellular, molecular and physiologic events. These events include vasodilatation; increased vascular permeability; extravasation of plasma leading to interstitial edema; chemotaxis of neutrophils, macrophages and lymphocytes; cytokine production; increased acute phase reactants; leukocytosis; fever; increased metabolic rate; impaired albumin production and hypoalbuminemia; activation of complement; and stimulation of antibodies.

Inflammation is associated with many different diseases or disorders such as, for example, neurodegenerative diseases, diabetes-associated nephropathy and retinopathy, protein wasting, muscle fatigue or inflammation, infectious diseases, various cardiovascular diseases or disorders including atherosclerosis, neurodegenerative diseases such as Alzheimer's disease, infectious disease such as, for example, myocarditis, cardiomyopathy, acute endocarditis, and pericarditis, Systemic Inflammatory Response Syndrome (SIRS)/sepsis, adult respiratory distress syndrome (ARDS), asthma, rheumatoid arthritis, osteoarthritis, systemic lupus erythematosis, airway hyperresponsiveness (AHR), bronchial hyperreactivity, chronic obstructive pulmonary disease (COPD), congestive heart failure (CHF), inflammatory complications of diabetes mellitus, metabolic syndrome, end stage renal disease (ESRD), and a variety of dermal conditions. Inflammation also results from injury to tissue.

A number of proximal mediators of the inflammatory response have been identified. In particular, C-reactive protein (CRP) has recently gained recognition as a marker for inflammatory conditions (see U.S. Pat. No. 6,040,147). CRP is an acute phase protein produced primarily in the liver in response to stimulation by IL6. It has also been identified as a marker of inflammation. Recent evidence has shown that CRP is also a risk factor for cardiovascular disease and stroke where inflammation plays an important role (Lagrand, W. K., et al, Circulation, 100: 96-102, 1999). In addition, elevated levels of CRP are associated with infectious diseases, such as, myocarditis, cardiomyopathy, acute endocarditis, or pericarditis; systemic inflammatory response syndrome (SIRS); diabetes; metabolic syndrome, as well as other forms of systemic inflammation. In humans, CRP levels are elevated in response to infection, trauma, surgery, tissue infarction, and in insulin-dependentdiabetes (IDDM) patients without macrovascular disease. The magnitude of the increase varies from about 50% to as much as 100-fold during systemic inflammation (Gabay, C., et al., New Engl. J. Med., 340:448-454, 1999). Most CRP production is from hepatocytes in response to pro-inflammatory cytokines, especially interleukin-6 and 1β (Ganter, U., et al., EMBO J., 8: 3773-3779, 1989), although macrophages have also been reported to release CRP (Dong, Q, et al, J. Immunol., 156: 4815-4820, 1996).

A mixture of high purity, high molecular weight straight chain aliphatic alcohols (collectively referred to herein as policosanol) has garnered much interest in recent years as a natural supplement for its cholesterol-lowering effects (Gouni-Berthold, et al., Am. Heart J., 143(2):356-365 (2002)). The main constituents of policosanol are tetracosanol, hexacosanol, octacosanol, and triacontanol, while eicosanol, docosanol, heptacosanol, nonacosanol, dotriacontanol, tetratriacontanol, and hexatriacontanol make up the remaining minor constituents of the straight chain aliphatic alcohols. There is a significant body of evidence demonstrating the benefits of policosanol with respect to cardiovascular disease. In the mid to late nineties, one research group proposed that policosanol was able to reduce endothelial damage by inhibiting the production of foam cells (Noa, et al., J. Pharm. Pharmacol., 48(3):306-309 (1996); Noa, et al., J. Pharm. Pharmacol., 49(10):999-1002 (1997)). Foam cells are macrophages that can migrate into the endothelium of the blood vessels and contribute to atherosclerotic plaque formation (Physicians' Desk Reference, 50 ed., Montvale, N.J.: Medical Economics Company; 2002.). Other researchers believe policosanol has a modulating effect on HMG-CoA reductase, the rate-controlling enzyme in cholesterol biosynthesis, but the precise mechanism remains unclear (Menendez, et al., Biol. Res., 27(3-4):199-203 (1994); Menendez, et al., Biol. Res., 29(2):253-257 (1996); and Menendez, et al., Arch. Med. Res., 32(1):8-12 (2001)). Still other investigators believe policosanol may inhibit cholesterol synthesis in the liver at a step before mevalonate production, but total inhibition of the HMG-CoA reductase is doubtful (Gouni-Berthold, et al., Am. Heart J., 143(2):356-365 (2002)). More recent work suggests policosanol inhibits LDL cholesterol oxidation (Menendez, et al., Can. J. Physiol. Pharmacol., 80(1):13-21 (2002); Menendez, et al., Br. J. Clin. Pharmacol., 50(3):255-262 (2000)). This was revealed when markers of peroxidation, such as thiobarbituric acid reactive substances (TBARS), and malondialdehyde (MDA) were lower in the cultures treated with policosanol. Oxidation of LDL cholesterol has been linked to heart disease and was the recent cover story in Scientific American magazine (Physicians' Desk Reference, 50 ed., Montvale, N.J.: Medical Economics Company; 2002).

There remains a need for effective compositions and methods for treating and/or ameliorating the symptoms of inflammation. Further, in view of risk factors associated with CRP, there is a need for methods for reducing elevated CRP levels associated with inflammation and inflammatory conditions, such as those described herein.

### SUMMARY OF THE INVENTION

The present invention relates to compositions and methods for the treatment and/or amelioration of inflammatory conditions and their associated systemic inflammatory response(s) and/or the reduction of CRP levels in a subject. The compositions of the present invention comprises a mixture of high purity, high molecular weight straight chain primary aliphatic alcohols, wherein the compositions comprise from about 1% to about 90% by weight policosanol. The compositions may further comprise from 0% to about 65% by weight of pharmaceutically acceptable formulation aids including, but not limited to diluents, stabilizers, binders, buffers, lubricants, coating agents, preservatives, emulsifiers and/or suspension agents. In one embodiment of this invention, the policosanol comprises at least one high molecular weight straight chain primary aliphatic alcohol selected from 20 to 36 carbon atoms.

The daily dosage is preferably between 1 and 100 mg of policosanol (more preferably between about 3 and 20 mg) and is intended for administration in any type or form.

In another embodiment, the policosanol composition of this invention comprises 1-tetracosanol, 1-hexacosanol, 1-heptacosanol, 1-octacosanol, 1-triacontanol, 1-dotriacontanol and 1-tetratriacontanol.

In yet another embodiment, the composition of the present invention comprises policosanol having the following quantitative composition:

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 0-30% |
| 1-hexacosanol (C₂₆) | 5-30% |
| 1-heptacosanol (C₂₇) | 0-5% |
| 1-octacosanol (C₂₈) | 5-80% |
| 1-nonacosanol (C₂₉) | 0-5% |
| 1-triacontanol (C₃₀) | 5-40% |
| 1-dotriacontanol (C₃₂) | 1-25% |
| 1-tetratriacontanol (C₃₄) | 0-7% |
| 1-hexatriacontanol (C₃₅) | 0-5% . |

The present invention further provides methods for using the compositions of this invention in the treatment and/or amelioration of a symptom of inflammation or a symptom of an inflammatory condition and/or for reducing the level of C-reactive protein (CRP). In one embodiment, the present invention provides compositions and methods for reducing CRP, thereby ameliorating an inflammatory symptom associated with disease or an inflammatory condition and/or reducing a subject's risk of progressing into long term or chronic inflammatory conditions. The present invention also provides compositions and methods for maintaining normal or healthy levels of CRP in a subject.

The present invention further provides kits having one or more containers comprising the therapeutic composition of the present invention and a suitable excipient as described herein and a set of instructions, generally written instructions although electronic storage media (e.g., magnetic diskette or optical disk) containing instructions are also acceptable, relating to the use and dosage of the therapeutic composition of the present invention for the intended treatment. The instructions included with the kit generally include information as to dosage, dosing schedule, and route of administration for the intended treatment. The containers of the therapeutic composition of the present invention may be unit doses, bulk packages (e.g., multi-dose packages) or sub-unit doses.

Inflammatory conditions that can be addressed by compositions and methods of the present invention include, but are not limited to, neurodegenerative diseases, diabetes-associated nephropathy and retinopathy, protein wasting, muscle fatigue or inflammation, infectious diseases, as well as various cardiovascular diseases or disorders, including atherosclerosis; neurodegenerative diseases such as, Alzheimer's disease; infectious disease, such as, for example, myocarditis, cardiomyopathy, acute endocarditis, pericarditis; Systemic Inflammatory Response Syndrome (SIRS)/sepsis; adult respiratory distress syndrome (ARDS); asthma; rheumatoid arthritis, osteoarthritis, systemic lupus erythematosis; airway hyperresponsiveness (AHR); bronchial hyperreactivity; chronic obstructive pulmonary disease (COPD); congestive heart failure (CHF); inflammatory complications of diabetes mellitus; metabolic syndrome, end stage renal disease (ESRD); as well as a variety of dermal conditions.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### DETAILED DESCRIPTION OF THE INVENTION

The present invention relates to compositions and methods for the treatment and/or amelioration of inflammatory conditions and their associated systemic inflammatory response(s) and/or the reduction of CRP levels in a subject. The compositions of the present invention comprises a mixture of high purity, high molecular weight straight chain primary aliphatic alcohols, wherein the composition comprises from about 1% to about 90% by weight policosanol.

The policosanol used in the present invention can be derived from any suitable source, each source being associated with a policosanol of particular characteristics, usually in terms of the relative proportions of its primary aliphatic alcohol components. For example, policosanol can be extracted and purified from a wide array of starting materials, such as, but not limited to, pela bug, natural waxes such as but not limited to, beeswax, carnauba wax, and candellia wax; bee pollen; oils such as but not limited to, peanut oil, sesame oil, cod liver oil, rice bran oil, oat oil, and rosemary needles oil; and powders such as but not limited to rice bran, containing primarily natural esters of aliphatic alcohols with caboxylic acids. Consequently, the quantitative compositions of policosanol can vary depending on the extraction process and starting materials that are used in its production. In one embodiment, the policosanol has the quantitative composition set forth in Table 1.

TABLE I

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 0-30% |
| 1-hexacosanol (C₂₆) | 5-30% |
| 1-heptacosanol (C₂₇) | 0-5% |
| 1-octacosanol (C₂₈) | 5-80% |
| 1-nonacosanol (C₂₉) | 0-5% |
| 1-triacontanol (C₃₀) | 5-40% |
| 1-dotriacontanol (C₃₂) | 1-25% |
| 1-tetratriacontanol (C₃₄) | 0-7% |
| 1-hexatriacontanol (C₃₅) | 0-5% |

U.S. Pat. Nos. 5,663,156; 5,856,316; 6,197,832; 6,225,354; and 6,596,776, all of which are incorporated herein by reference, disclose policosanol compositions that are specific to the starting material and extraction processes used. It should be noted that while any commercially available policosanol or any of the policosanols disclosed in the above-referenced patents are suitable for use in the present invention, for purposes of the remainder of this discussion the policosanol and methodologies disclosed in U.S. Pat. No. 6,596,776 will be referenced. Specifically, in one embodiment the policosanol used in the present invention is obtained from beeswax and has the formulation set forth in Table II.

TABLE II

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 13-28% |
| 1-hexacosanol (C₂₆) | 5-30% |
| 1-heptacosanol (C₂₇) | 0-5% |
| 1-octacosanol (C₂₈) | 15-25% |
| 1-triacontanol (C₃₀) | 25-40% |
| 1-dotriacontanol (C₃₂) | 5-15% |
| 1-tetratriacontanol (C₃₄) | 0-5% |

The process used to isolate the policosanol composition described in Table II is incorporated herein by reference and is briefly described as follows. Beeswax is initially subjected to a homogenous phase saponification step, after which the saponified beeswax is dried and ground to a particle mesh size of 100-500 microns. Alternatively, unsaponified beeswax of varying purity may be used as the starting material and is initially dried and ground to a particle mesh size of 100-200 microns. The particles of saponified or unsaponified beeswax are placed into a conventional solid-liquid extractor and a hot organic solvent is introduced and contacted with the beeswax particles. The suspension is mixed and then hot-filtered to remove any solids.

The resulting extract is then maintained within the temperature range of 2° C. to 10° C., causing the aliphatic alcohols to solidify and form a suspension. The suspension is filtered and the first solids are recovered and air dried. The dried solids are then sent to a purifier where they are contacted with and dissolved in a second hot solvent and hot-filtered. This solution is then cooled and the second solids are collected and dried under vacuum. The dried solids obtained from the second purification step are contacted with a third hot organic solvent, which dissolves the solids. This solution is hot-filtered and chilled, and the third solids are collected, dried, and powdered to provide the final product disclosed in Table II.

After the particles are dried, they can be formulated into a conventional pharmaceutical formulation for administration. The formulations of the present invention comprise a therapeutically effective amount policosanol effective to reduce inflammation and/or symptoms due to inflammation and/or reduce levels of CRP in a subject. For example, the policosanol can be present in a range between about 0.5 and 99%.

By “amount effective to reduce inflammation and/or symptoms due to inflammation” is meant that policosanol (alone or together with an additional anti-inflammatory agent) is administered in a sufficient dose or to achieve a final concentration sufficient for reducing inflammation, as measured by a reduction of CRP blood levels in the subject, and/or reduction of symptoms associated with inflammation, such as for example, pain and/or edema associated with inflammation. This amount includes, but is not limited to, a concentration which acts as a prophylaxis or treatment for a symptom of inflammation. Thus, an “effective amount” is an amount sufficient to effect beneficial or desired results. An effective amount can be administered in one or more administrations. For purposes of this invention, an effective amount of policosanol is an amount that is sufficient to ameliorate, stabilize, reverse, slow or delay the progression of injury(ies) in subjects i) at risk for or suffering from a disease, disorder or condition associated with inflammation or ii) associated with, due to and/or symptoms of inflammation. An effective amount also includes an amount that is able to reduce CRP levels in a subject. Preferably, amelioration of symptoms due to inflammation can be quantified by an assay measuring, for example, reduction in CRP levels.

The term “amelioration” includes, but is not limited to, the prevention, reduction or palliation of a state, or improvement of the state of a subject. Amelioration includes, but does not require complete recovery or complete prevention of a symptom or condition. More specifically, amelioration may be considered to be at least about 30%, at least about 50%, at least about 70%, at least about 80%, and at least about 90% reduction in the levels of inflammatory markers associated with inflammation or an inflammatory condition or a reduction in the symptoms associated with inflammation such as for example, pain and/or edema associated with inflammation.

The term “treatment” or “treating” includes, but is not limited to, any treatment of a disease or disorder, in a mammal, including, but not limited to: preventing or protecting against the disease or disorder that causes the clinical symptoms of the disease; inhibiting the disease, that is, arresting or suppressing the development of clinical symptoms; and/or relieving the disease, that is, causing the regression of clinical symptoms.

As used herein, “inflammation” or “inflammatory symptoms” refers to one or more biological and physiological sequelae including, but not limited to: vasodilatation; increased vascular permeability; extravasation of plasma leading to interstitial edema; chemotaxis of neutrophils, macrophages and lymphocytes; cytokine production; acute phase reactants; C-reactive protein (CRP); increased erythrocyte sedimentation rate; leukocytosis; fever; increased metabolic rate; impaired albumin production and hypoalbuminemia; activation of complement; and stimulation of antibodies.

Inflammation is associated with a number of diseases, disorders and conditions such as for example, cardiovascular diseases or disorders; neurodegenerative diseases such as, Alzheimers; infectious diseases, such as, for example, myocarditis, cardiomyopathy, acute endocarditis, pericarditis; atherosclerosis; Systemic Inflammatory Response Syndrome (SIRS)/sepsis; adult respiratory distress syndrome (ARDS); asthma; rheumatoid arthritis, osteoarthritis, systemic erythematosis (SLE); Airway hyperresponsiveness (AHR); bronchial hyperreactivity; Chronic Obstructive Pulmonary disease (COPD); Congestive Heart Failure (CHF); inflammatory complications of diabetes mellitus; metabolic syndrome; end-stage renal disease (ESRD); muscle fatigue or inflammation and dermal conditions. As used herein, the foregoing listed conditions, and any conditions that have as a symptom inflammation, are encompassed by the term “systemic inflammatory condition” or “inflammatory condition.” As used herein, “respiratory inflammatory conditions” refer to inflammatory conditions that primarily affect the lungs, for example, SIRS, ARDS, asthma and AHR.

Elevated levels of C-reactive protein (CRP) have been associated with various inflammatory conditions. As used herein, “CRP-associated inflammation” refers to inflammatory conditions and/or inflammation associated with elevated levels of CRP such as for example, cardiovascular diseases or disorders, including atrial fibrillation, unstable angina, coronary artery disease, peripheral artery disease, cardiac allograft vasculopathy (CAVD); mastitis; pre-eclampsia; inflammatory bowel conditions; stroke; tissue infarction; lumbosciatica; estrogen/progestin hormone replacement therapy (HRT); infection (bacterial, viral and protozoan); bacterial meningitis; trauma; surgery; biomaterial implants; smoking; obesity; neurodegenerative diseases such as, Alzheimers; infectious disease, such as, for example, myocarditis, cardiomyopathy, acute endocarditis, pericarditis; atherosclerosis; SIRS/sepsis; adult respiratory distress syndrome ARDS; asthma; rheumatoid arthritis, osteoarthritis, systemic lupus erythematosis (SLE); AHR; bronchial hyper-reactivity; COPD; CHF; inflammatory complications of diabetes mellitus type I and type II; metabolic syndrome; end stage renal disease (ESRD), muscle fatigue or inflammation; multiple organ dysfunction syndrome (MODS); aging; acute allergic reactions; gingivitis and dermal conditions.

As used herein, “cardiovascular disease” includes diseases associated with the cardio-pulmonary and circulatory systems including but not limited to ischemia, angina, edematous conditions, artherosclerosis, CHF, LDL oxidation, adhesion of monocytes to endothelial cells, foam-cell formation, fatty-streak development, platelet adherence, and aggregation, smooth muscle cell proliferation, reperfusion injury, high blood pressure, and thrombolic disease.

As used herein, a “symptom” of an inflammatory condition includes, but is not limited to, physical symptoms (pain, edema, erythema, and the like) associated with a particular inflammatory condition.

A “subject” includes both humans and other animals (particularly mammals) that receive either prophylactic or therapeutic treatment according to this invention.

Policosanol is extremely well tolerated. In animal toxicity studies, doses up to 500 mg/kg/day, a dose that is 1500 times the normal human dose of 20 mg/day, have shown no negative effects on carcinogenesis, reproduction, growth, or development. In one embodiment, a total dose of policosanol administered to a subject according to the present invention is in the range from about 1 mg to 100 mg per day. In another embodiment, a total dose of policosanol between about 5 mg to 40 mg per day is administered. In yet another embodiment, a total dose of policosanol between about 10 to 20 mg per day is administered.

The pharmaceutical formulations of the present invention can contain as an active ingredients from about 0.5 to about 95.0 wt % of policosanol. This dosage is obtained by mixing the policosanol with one or more excipients, including, but not limited to agglutinants, disintegrators, lubricants, sliders or fillers. For example, specific excipients include, but are not limited to, lactose, corn starch, saccharose, magnesium stearate, microcrystalline cellulose, sodium croscarmellose gelatin, cellulose acetophtalate, titanium dioxide, special talc for tablets and polyethylene glycol. Other suitable excipients are well known in the art. The formulation may further comprise from 0% to about 65% by weight of pharmaceutically acceptable formulation aids, including but not limited to diluents, stabilizers, binders, buffers, lubricants, coating agents, preservatives, emulsifiers and/or suspension agents.

In some embodiments, compositions are administered in one dosing of a single formulation and in other embodiments, compositions are administered in multiple dosing of a single formulation.

A composition of the present invention may further be used in combination with other methods known in the art for treating or ameliorating inflammation. For example, a method according to this invention can include administering policosanol and at least one anti-inflammatory agent as an admixture, separately and simultaneously, or separately in any order. Additional anti-inflammatory agents that can be used in conjunction with the administration of policosanol according to this invention include compounds, mixtures, or formulations of compounds which are capable of treating or ameliorating the symptoms of inflammation, such as by reducing the levels of inflammatory markers, e.g., CRP, cytokines associated with inflammation, such as members of the interleukin family, including IL-1 through 17, TNF-alpha; B61; certain cellular adhesion molecules, such as for example, e-selectin (also known as ELAM), sICAM, integrins, ICAM-1, ICAM-3, BL-CAM, LFA-2, VCAM-1, NCAM and PECAM; neopterin; serum procalcitonin; leukotriene, thromboxane, isoprostane and/or by reducing pain and/or edema associated with the inflammation.

The amount of policosanol in the formulations of this invention that is combined with one or more excipients to produce a single dosage form will necessarily vary depending upon the host treated and the particular route of administration. For example, a formulation intended for oral administration to humans may contain, for example, from 1-30 g of policosanol compounded with an appropriate and convenient amount of excipients which may vary from about 5 to about 95 percent by weight of the total composition. Dosage unit forms will generally contain about 1-30 g of policosanol. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In order to use the compositions of this invention for the therapeutic treatment (including prophylactic treatment) of a subject in need thereof according to the methods of this invention, policosanol is formulated in accordance with standard pharmaceutical practice as a pharmaceutical composition as discussed above. According to this aspect of the invention there is provided a pharmaceutical formulation comprising policosanol in association with a pharmaceutically acceptable diluent or carrier, wherein the policosanol is present in an amount for effectively treating or ameliorating inflammatory conditions and their associated systemic inflammatory response(s) and/or reducing CRP levels in an subject.

The formulations of the present invention can be administered to a subject by any available and effective delivery system including, but not limited to, parenteral, transdermal, intranasal, sublingual, transmucosal, intra-arterial, or intradermal modes of administration in dosage unit formulations containing conventional nontoxic pharmaceutically acceptable carriers, adjuvants, and in a vehicle as desired, such as a depot or a controlled release formulation.

For example, a pharmaceutically acceptable formulation of the composition of the present invention may be formulated for parenteral administration, e.g., for intravenous, subcutaneous, or intramuscular injection. For an injectable formulation, policosanol may be combined with a sterile aqueous solution which is preferably isotonic with the blood of the subject. Such a formulation may be prepared by dissolving a solid active ingredient in water containing physiologically-compatible substances such as sodium chloride, glycine, and the like, and having a buffered pH compatible with physiological conditions so as to produce an aqueous solution, and then rendering the solution sterile by methods known in the art. The formulations may be present in unit or multi-dose containers, such as sealed ampules or vials. The formulation may be delivered by any mode of injection, including, without limitation, epifascial, intracutaneous, intramuscular, intravascular, intravenous, parenchymatous, subcutaneous, oral or nasal preparations (see, for example, U.S. Pat. No. 5,958,877, which is specifically incorporated herein by reference).

The compositions of this invention may also be in the form of a sterile injectable aqueous or oily suspension, which may be formulated according to known procedures using one or more appropriate dispersing or wetting agents and suspending agents. A sterile injectable preparation may also be a sterile injectable solution or suspension in a non-toxic parenterally-acceptable diluent or solvent, for example a solution in 1,3-butanediol.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing or as a suppository for rectal dosing), or as vehicles such as a depot or other controlled release formulation.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which policosanol is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain policosanol in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending policosanol in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, wherein policosanol may be formulated according to known procedures using one or more of the appropriate dispersing or wetting agents and suspending agents, which have been described herein. A sterile injectable preparation may also be a sterile injectable solution or suspension in a non-toxic parenterally-acceptable diluent or solvent, for example a solution in 1,3-butanediol.

Suppository formulations may be prepared by mixing policosanol with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating policosanol with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either policosanol alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense policosanol either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

Controlled Release Formulations

Another aspect of this invention provides methods of treating and/or ameliorating inflammatory conditions and their associated systemic inflammatory response(s) and/or reducing CRP levels in a subject as a controlled release formulation. As used herein, the terms “controlled” “extended” “sustained” or “prolonged” release formulations and methods of the present invention will collectively be referred to as “controlled release” formulations and methods, and include continuous and discontinuous, linear and non-linear release of the composition of the present invention. There are many advantages for a controlled release formulation. Among these are to effectively treat the subject during a period when the subject would not be readily able or willing to periodically ingest the composition of the present invention. The single dose of composition of the present invention preferably is administered via ingestion of one or more controlled release unit dosage forms, so that effective policosanol levels are maintained.

1. Tablets

A useful controlled release tablet is disclosed in U.S. Pat. No. 5,126,145 which is incorporated herein by reference. This tablet comprises, in admixture, about 5-30% high viscosity hydroxypropyl methyl cellulose, about 2-15% of a water-soluble pharmaceutical binder, about 2-20% of a hydrophobic component such as a waxy material, e.g., a fatty acid, and about 30-90% policosanol.

More specifically, one such useful controlled release tablet comprises: (a) about 5-20 percent by weight hydroxypropyl methylcellulose having a viscosity of about 10,000 CPS or greater, a substitution rate for the methoxyl group of about 7-30% and a substitution rate for the hydroxypropoxyl group of about 7-20%; (b) about 2-8 percent hydroxypropyl methylcellulose having a viscosity of less than about 100, CPS methyl cellulose, or polyvinyl pyrollidone; (c) about 5-15 percent by weight hydrogenated vegetable oil or stearic acid; and (d) about 30-90% policosanol.

High viscosity water-soluble 2-hydroxypropyl methyl cellulose (HPMC) is particularly preferred for use in the present tablets and in the controlled-release tablet coating, due to its sustaining properties with respect to policosanol release. A particularly preferred high viscosity HMPC has a nominal viscosity, two percent solution, of about 100,000 CPS, methoxyl content of about 19-24, a hydroxypropyl content of about 7-12 percent, and a particle size where at least 90% passes through a USS 100 mesh screen. (Methocel® K100MCR). Low viscosity HPMC is preferred as the binder component of the tablet. A particularly preferred low viscosity HPMC has a methoxyl content of about 20-30%, a hydroxylpropyl content of about 7-12 percent, and a particle size where 100% will pass through a USS No. 30 mesh screen and 99% will pass through a USS 40 mesh screen (Methocel® EIS). In some cases, a portion of the high viscosity HPMC can be replaced by a medium viscosity HPMC, i.e., of about 2000-8,000 cps.

The viscosities reported herein are measured in centipoises (cps or cP), as measured in a 2% by weight aqueous solution of the cellulose either at 20° C. using a rotational viscometer. A “high viscosity” cellulose ether possesses a viscosity of at least about 10,000 cps i.e., about 50,000-100,000 cps. A low-viscosity cellulose ether possesses a viscosity of less than about 100 cps, i.e., about 10-100 cps.

“Water soluble” for purposes of this application means that two grams of powdered cellulose ether can be dispersed by stirring into 100 grams of water at a temperature between 0° C. and 100° C. to provide a substantially clear, stable aqueous composition or dispersion (when the dispersion is brought to 20° C.).

Useful hydrophobic components include natural and synthetic waxes such as beeswax, carnauba wax, paraffin, spermaceti, as well as synthetic waxes, hydrogenated vegetable oils, fatty acids, fatty alcohols and the like.

The controlled release policosanol tablets preferably can be formulated to contain 10 mg, 20 mg or 40 mg of policosanol, and are ingested orally. Preferably, these tablets will release about 10-35 wt % of the total policosanol within about 2 hours in an in vitro dissolution test, and about 40-70 wt % of the total policosanol in eight hours.

2. Films

This invention further provides a method of treating and/or ameliorating inflammatory conditions and their associated systemic inflammatory response(s) and/or reducing CRP levels in a subject, comprising administering to a subject a biodegradable, biocompatible polymeric film comprising policosanol. The polymeric films are thin compared to their length and breadth. The films typically have a uniform selected thickness between about 60 micrometers and about 5 mm. Films of between about 600 micrometers and 1 mm and between about 1 mm and about 5 mm thick, as well as films between about 60 micrometers and about 1000 micrometers; and between about 60 and about 300 micrometers are useful in the manufacture of therapeutic implants for insertion into a subject's body. The films can be administered to the subject in a manner similar to methods used in adhesion surgeries. For example, a policosanol film formulation can be sprayed or dropped onto a tissue site during surgery, or a formed film can be placed over the selected tissue site. In an alternative embodiment, the film can be used as controlled release coating on a medical device such as a stent.

Either biodegradable or nonbiodegradable polymers may be used to fabricate implants in which the policosanol is uniformly distributed throughout the polymer matrix. A number of suitable biodegradable polymers for use in making the biodegradable films of this invention are known to the art, including polyanhydrides and aliphatic polyesters, preferably polylactic acid (PLA), polyglycolic acid (PGA) and mixtures and copolymers thereof, more preferably 50:50 copolymers of PLA:PGA and most preferably 75:25 copolymers of PLA:PGA. Single enantiomers of PLA may also be used, preferably L-PLA, either alone or in combination with PGA. Polycarbonates, polyfumarates and caprolactones may also be used to make the implants of this invention.

A plasticizer may be incorporated in the biodegradable film to make it softer and more pliable for applications where direct contact with a contoured surface is desired.

The polymeric films of this invention can be formed and used as flat sheets, or can be formed into three-dimensional conformations or “shells” molded to fit the contours of the tissue site into which the film is inserted.

To make the polymeric films of this invention, a suitable polymeric material is selected, depending on the degradation time desired for the film. Selection of such polymeric materials is known to the art. A lower molecular weight, e.g., around 20,000 daltons, 50:50 or 55:45 PLA:PGA copolymer is used when a shorter degradation time is desired. To ensure a selected degradation time, the molecular weights and compositions may be varied as known to the art.

Polymeric films of this invention may be made by dissolving the selected polymeric material in a solvent known to the art, e.g., acetone, chloroform or methylene chloride, using about 20 mL solvent per gram of polymer. The solution is then degassed, preferably under gentle vacuum to remove dissolved air and poured onto a surface, preferably a flat non-stick surface such as BYTAC (Trademark of Norton Performance Plastics, Akron, Ohio) non-stick coated adhesive-backed aluminum foil, glass or TEFLON™ non-stick polymer. The solution is then dried, preferably air-dried, until it is no longer tacky and the liquid appears to be gone. The known density of the polymer may be used to back-calculate the volume of solution needed to produce a film of the desired thickness.

Films may also be made by heat pressing and melt forming/drawing methods known to the art. For example, thicker films can be pressed to form thinner films, and can be drawn out after heating and pulled over forms of the desired shapes, or pulled against a mold by vacuum pressure.

The amount of policosanol to be incorporated into the polymeric films of this invention is an amount effective to show a measurable effect in reducing CRP levels. The composition of the present invention can be incorporated into the film by various techniques such as by solution methods, suspension methods, or melt pressing.

Solid implants comprising policosanol can also be made into various shapes other than films by injection molding or extrusion techniques. For example, the implant can comprise a core material such as ethylene/vinyl acetate copolymer, and a vinyl acetate content of 20% by weight or more and which functions as a matrix for the policosanol, in a quantity which is sufficient for a controlled release of policosanol, and a membrane which encases the core material and also consists of EVA material and an acetate content of less than 20% by weight. The implant can be obtained, for example, by means of a co-axial extrusion process, a method in which the two EVA polymers are extruded co-axially with the aid of a co-axial extrusion head. The co-axial extrusion process is well known in the art known and need not be described further.

3. Transdermal Patch Device

Transdermal delivery involves delivery of a therapeutic agent through the skin for distribution within the body by circulation of the blood. Transdermal delivery can be compared to continuous, controlled intravenous delivery of a drug using the skin as a port of entry instead of an intravenous needle. The therapeutic agent passes through the outer layers of the skin, diffuses into the capillaries or tiny blood vessels in the skin and then is transported into the main circulatory system.

Transdermal patch devices that can be used to provide a controlled, continuous administration of policosanol through the skin are well known in the art. Such devices, for example, are disclosed in U.S. Pat. Nos. 4,627,429; 4,784,857; 5,662,925; 5,788,983; and 6,113,940, which are all incorporated herein by reference. Characteristically, these devices contain a drug-impermeable backing layer which defines the outer surface of the device and a permeable skin-attaching membrane, such as an adhesive layer, sealed to the barrier layer in such a way as to create a reservoir between them in which the therapeutic agent is placed. In one embodiment of the present invention, policosanol is introduced into the reservoir of a transdermal patch.

4. Kits

The therapeutic composition of the present invention may be packaged in any convenient, appropriate packaging. The present invention therefore further provides kits having one or more containers comprising the therapeutic composition of the present invention and a suitable excipient as described herein and a set of instructions, generally written instructions although electronic storage media (e.g., magnetic diskette or optical disk) containing instructions are also acceptable, relating to the use and dosage of the therapeutic composition of the present invention for the intended treatment. The instructions included with the kit generally include information as to dosage, dosing schedule, and route of administration for the intended treatment. The containers of the therapeutic composition of the present invention may be unit doses, bulk packages (e.g., multi-dose packages) or sub-unit doses.

Inflammatory Conditions

The present invention includes methods and formulations for treating and/or ameliorating inflammation and symptoms of inflammatory conditions. The invention is particularly directed at reducing the level of CRP in a subject, which has been implicated as an adverse prognostic indicator of subsequent complications of such conditions. This section will describe exemplary inflammatory conditions that may be improved by administration of formulations of the present invention.

Inflammation is associated with a number of conditions, including cardiovascular diseases or disorders; neurodegenerative diseases such as, Alzheimers; infectious disease, such as, for example, myocarditis, cardiomyopathy, acute endocarditis, pericarditis; atherosclerosis; SIRS/sepsis; ARDS; asthma; rheumatoid arthritis, osteoarthritis, SLE; AHR; bronchial hyperreactivity; COPD, CHF; inflammatory complications of diabetes mellitus; metabolic syndrome; end stage renal disease (ESRD); muscle fatigue or inflammation; and dermal conditions. While the particular symptoms and complications may vary, the conditions discussed in detail below are exemplary of inflammatory conditions that are amenable to treatment by formulations of the present invention.

Muscle Inflammation. Inflammation can be induced by acute exercise in untrained individuals (Jenkins, et al., 1993, Med. Sci. Sports Exerc. 25: 213-217). Inflammation stimulates polymorphonuclear leukocytes and macrophages that produce large amounts of lipid peroxidation products. These peroxidation products are postulated to cause significant damage to DNA and to several other biomolecules in vivo, including, enzymes and lipid membranes (Leeuwenburgh et al. 1999, Free Radic. Biol. Med. 27:186-192; Powers et al., 1999, Med. Sci. Sports Exerc. 31:987-997; Fielding et al., 2000, Med. Sci. Sports Exerc. 32:359-64). In addition, lipid peroxidation products are potentially involved in the actual tissue damage associated post-exercise muscle injury.

Eccentric exercise presents an acute condition characterized by severe inflammation (MacIntyre et al., 2000, Eur. J Physiol. 81:47-53). An example of an eccentric exercise is weight resistance arm exercises, which have been shown to increase neutrophil migration into the skeletal muscle after such injury (Fielding et al., supra, MacIntyre et al., supra). Severe inflammation, pain, and a decrease in range of motion characterize this type of injury.

Diabetes. Diabetes mellitus is a chronic disorder affecting carbohydrate, fat and protein metabolism. Of the two major forms of diabetes, one form, insulin-dependent diabetes (IDDM) (also known as insulin-sensitive diabetes, type I or juvenile diabetes) is caused by under-production of insulin in the pancreas. By far the most prevalent form of diabetes is so-called “adult-onset” or Type II diabetes (non-insulin dependent diabetes, NIDDM). Secondary diabetes is associated with other conditions, including pancreatic disease (e.g. chronic pancreatitis), endocrine diseases (e.g. acromegaly or Cushing's disease), and certain medications or toxins (e.g. thiazides, glucocorticoids). Polycystic ovary syndrome is also associated with elevated insulin levels, insulin resistance or diabetes. Gestational diabetes includes glucose intolerance with the onset of pregnancy, usually at 24-30 weeks gestation (Nathan 1993 Ch. 9(IV) in Scientific American Medicine, Rubenstein & Federman, eds., Scientific American, Inc., New York).

All forms of diabetes are characterized by end-organ damage in later phases of the disease, and it is now becoming apparent that inflammation contributes to this aspect of the condition. Without being bound to a particular mechanistic theory, one possible factor is accelerated production of reactive oxygen species known to occur in diabetics, and in particular the increased formation of isoprostanes, which may impair insulin action via reduction of membrane arachidonate and increased inflammatory mediators.

The long term complications of diabetes include numerous vascular conditions, macrovascular, microvascular, and neurologic secondary diabetes and gestational diabetes. While the etiology and ultimate causes of diabetes mellitus vary, the complications linked to the associated metabolic dysfunction and the complications which arise therefrom are common to all types. Common complications include microvascular, neurologic and macrovascular conditions. Complications such as retinopathy and nephropathy are specific for diabetes. Nephropathy associated with diabetes may lead to pre-end stage renal disease (ESRD) and ESRD.

End-Stage Renal Disease (ESRD). ESRD is associated with known cardiovascular disease risk factors such as hypertension, hyperlipidemia and diabetes mellitus. Cardiovascular mortality rate in dialysis patients is 10 to 20 times higher than the general population, and the 5-year survival rate after initiation of hemodialysis is less than 50%. Recently, attention has been focused on evidence that dialysis patients also have elevated circulating markers of oxidative stress and inflammation, both of which are associated with accelerated atherosclerosis. No therapeutic or preventive agents targeting reduction of these proposed risk factors has proven to reduce mortality in this at-risk population.

Clinical evidence of this effect of oxidative stress inducing increased in HbA1c independent of blood glucose level comes from end-stage renal disease, where increased levels of HbA1c have been observed in non-diabetic patients. In addition, such subjects may experience increased levels of pre-albumin and albumin.

Metabolic Syndrome. Insulin resistance is a common characteristic underlying multiple cardiovascular disease (CVD) risk factors, including hypertension, dyslipidemia, and obesity, as well as type-2 diabetes. Metabolic syndrome can be considered to be an inflammatory condition, and is now defined as a convergence of these predictors—specifically at least three of the following (blood pressure>130/>85 mm Hg; triglyceride>150 mg/dl, HDL-cholesterol<40 and<50 for men and women, respectively; waist>40 and>35 inches for men and women, respectively; fasting glucose>110 mg/dl). These risk factors for metabolic syndrome are highly concordant; in aggregate they enhance risk for CVD.

Cardiovascular Inflammation and Disease. Myocarditis and cardiomyopathy are a group of diseases primarily of the myocardium which do not result from hypertensive, congenital, ischemic, or valvular heart disease. These conditions result from an immune response against the myocardium, including lymphocytic infiltration and inflammation. This immune response can occur secondary to infectious diseases such as Chagas' disease (American trypanosomiasis), toxoplasmosis, trichinosis, ricksettal infection (typhus, Rocky Mountain spotted fever), fungal infections, and metazoan parasites; or secondary to autoimmune diseases such as rheumatic fever, rheumatoid arthritis, systemic lupus erythematosus, progressive systemic sclerosis, and polyarteritis nodosa. The immune response leading to myocarditis can be idiopathic in nature as seen in Fiedler's myocarditis. Additionally, myocarditis can be caused by drug reaction to penicillin or sulfonamide, for example. See U.S. Pat. No. 5,496,832. Myocarditis generally defines acute myocardial disease characterized by inflammation, and cardiomyopathy defines more chronic myocardial diseases in which the inflammatory features are not conspicuous. Myocarditis and cardiomyopathy can lead to fever, chest pain, leukocytosis, increased erythrocyte sedimentation rate, left ventricular failure, arrythmias, heart block, ECG changes, and eventually cardiac failure. See U.S. Pat. No. 5,496,832.

Acute pericarditis is defined as an inflammatory disease of the visceral or parietal pericardium and can occur secondary to bacterial, viral (especially echovirus, and Coxsackie Group B), or fungal infection, and can accompany systemic diseases such as rheumatoid arthritis, systemic lupus erythematosus, scleroderma, and uremia. Pericarditis can also occur after cardiac trauma or cardiac surgery that is suggested as being caused by immunologic hypersensitivity. Acute pericarditis can lead to chronic constrictive pericarditis, pericardial tamponade, effusion, and hemorrhage, all of which can result in cardiac failure. See U.S. Pat. No. 5,496,832.

Inflammation, particularly macrophage-mediated and chronic inflammation, has been cited as central to atherosclerosis (U.S. Pat. Nos. 5,877,203 and 6,210,877) and may serve as a prognostic marker for heightened risk of myocardial infarction (Boisvert et al. 1998 J. Clin. Invest. 101(2):353-363). Atherosclerosis (also known as arteriosclerosis) is the term used to described progressive luminal narrowing and hardening of the arteries. This disease process can occur in any of the arteries in the body leading to a variety of conditions including stroke (hardening or narrowing of arteries leading to the brain), gangrene (hardening or narrowing of peripheral arteries) and CAD (hardening or narrowing of arteries supplying the myocardium). CAD can in turn lead to myocardial ischemia or myocardial infarction. Cardiovascular disorders associated with atherosclerotic disease (and therefore inflammation) can include, for example, myocardial infarction, stroke, angina pectoris and peripheral arteriovascular disease. Macrovascular complications, including atherosclerosis and related conditions are often complications associated with diabetes and metabolic syndrome.

Luminal narrowing of the arteries is the result of the depositions of atheromatous plaque. The plaque consists of a mixture of inflammatory and immune cells, fibrous tissue and fatty material such as low density lipids (LDLs), modifications thereof and α-lipoprotein. The initial causes of atherosclerosis are not completely understood, but it has been suggested that the pathogenesis may include the following stages: endothelial cell dysfunction and/or injury; monocyte recruitment and macrophage formation; lipid deposition and modification; vascular smooth muscle cell proliferation; and synthesis of extracellular matrix. Recent reports have implicated CRP levels with increased risk of cardiovascular disease (Ridker, P. M., et al., N Engl. J. Med., 347(20): 1557-1565).

Respiratory Inflammatory Conditions. Trauma or infection may result in acute life-threatening conditions which include systemic inflammatory response syndrome (SIRS), or adult respiratory distress syndrome (ARDS). When SIRS is caused by infection, it is termed sepsis, which in turn has progressively severe stages (severe sepsis and septic shock). SIRS/sepsis may also result from numerous sources, including bacterial, viral, parasitic, rickettsial or fungal infection, and/or SIRS resulting from non-infectious causes such as burns, pancreatitis, multitrauma, severe surgical trauma, transplant rejection, marked autoimmune rejection, ischemia reperfusion, transfusion reaction or heat stroke. The marked augmentation of pro-inflammatory cytokines which leads to SIRS may also lead to multiple organ dysfunction syndrome (MODS) (e.g. varying degrees of fever, hypoxemia, tachypnea, tachycardia, endothelial inflammation, myocardial insufficiency, hypoperfusion, altered mental status, vascular collapse, which may lead to ARDS, coagulopathy, cardiac failure, renal failure, shock and/or coma).

Chronic asthma can be considered to be predominantly an inflammatory disease with associated bronchospasm. The degree of reactivity and narrowing of the bronchi in response to stimuli is greater in asthmatics than in normal individuals. Persistent inflammation is responsible for the bronchial hyperreactivity or airway hyperresponsiveness (AHR). Mucosal edema, mucus plugging and hypersecretion may also be present and pulmonary parenchyma is normal. Airway narrowing may reverse spontaneously or through treatment. Type 1 (immediate) immune responses may play an important role in the development of asthma in children and many adults; however, when onset of disease occurs in adulthood, allergic factors may be difficult to identify. Exposure to cold dry air, exercise and other aggravating factors may also trigger asthma.

Bronchial hyperreactivity (or airway hyperreactivity, AHR) is a hallmark of asthma and is closely related to underlying airway inflammation. Worsening of asthma and airway inflammation is associated with an increase in bronchial hyperreactivity, which can be induced by both antigenic and non-antigenic stimuli. At present, corticosteroids are one of the most effective agents available which diminish bronchial hyperreactivity. Although inhaled corticosteroids are relatively safe in adult patients with asthma, these agents have tremendous toxicity in children, including adrenal suppression and reduced bone density and growth.

While asthma was once thought of as a disease associated primarily with morbidity, it is now being recognized that asthma is more often associated with mortality than generally thought. In the United States, the annual mortality for asthma, among persons 5 to 34 years is 0.4 per 100,000 people. Deaths are most likely the result of asphyxiation caused by inadequately treated airflow obstruction and generally occur outside of the hospital (Leatherman et al., 1992 Ch. 14(II) in Scientific American Medicine Rubenstein, E. and Federman, D. D. eds. Scientific American, Inc., New York).

Inflammation is also associated with pulmonary or respiratory conditions other than asthma, including adult respiratory distress syndrome (ARDS), an acute and life threatening disease which can lead to multiple organ dysfunction (MOD) (U.S. Pat. No. 5,780,237), and chronic obstructive pulmonary disease (COPD) which is often a complication of cystic fibrosis (Kennedy 2001 Pharmacotherapy 215:593-603). ARDS is a classic example of a restrictive diffuse pulmonary disease while COPD and asthma are exemplary of an obstructive (or airway) disease. Obstructive diseases are characterized by an increase in resistance to air flow due to partial or complete obstruction, while restrictive diseases are characterized by reduced expansion of lung parenchyma and a decreased total lung capacity. COPD (also known as COAD, chronic obstructive airway disease) refers to a group of conditions, emphysema, chronic bronchitis, bronchial asthma and bronchiectasis, which are accompanied by chronic or recurrent obstruction to air flow within the lung (Cotran et al., “Robbins Pathologic Basis of Disease” 4th Ed. 1989, W. B. Saunders Co., Philadelphia, Pa.).

ARDS (also known as acute respiratory distress syndrome) is defined as respiratory failure in adults or children that results from diffuse injury to the endothelium of the lung (as in sepsis, chest trauma, massive blood transfusion, aspiration of the gastric contents, or diffuse pneumonia) and is characterized by pulmonary edema, respiratory distress and hypoxemia (Merriam-Webster's Medical Desk Dictionary 1996 Merriam-Webster, Inc. Springfield, Mass.). ARDS can be due to either trauma or infection and generally occurs a clinical setting. CF pulmonary disease is characterized as multi-factorial, involving a cycle of airway obstruction, chronic infection and excessive local inflammation that leads to development of bronchiectasis (Kennedy supra), which can be a chronic inflammatory or degenerative condition of the bronchi or bronchioles. Uncontrolled, chronic inflammation directly damages the airway wall, which leads to bronchiectasis and decline in pulmonary function.

The invention is further illustrated by the following non-limiting example. All scientific and technical terms have the meanings as understood by one with ordinary skill in the art. The specific examples which follow illustrate the methods in which the compositions of the present invention may be prepared and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### EXAMPLE 1

Tablets comprising a composition of the present invention are prepared as set out in Table III.

TABLE III

| Ingredient | amt/cap | | function |
|---|---|---|---|
| Policosanol | 20 | mg | active |
| Calcium phosphate | 261.7 | mg | base |
| Cellulose | 49.4 | mg | tablet coating agent |
| Stearic acid | 23.8 | mg | lubricant |
| Magnesium stearate | 6.8 | mg | lubricant |
| Silicon dioxide | 9.4 | mg | diluent |

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

The embodiments of the invention in which an exclusive property or privilege is claimed are defined as follows:

1. A method for treating or ameliorating a symptom of an inflammatory condition in a subject, comprising administering to the subject a therapeutically effective amount of a composition comprising policosanol.

2. The method of claim 1 wherein said policosanol comprises at least one higher primary aliphatic alcohol selected from straight chain primary aliphatic alcohols having 20 to 36 carbon atoms.

3. The composition of claim 2 wherein said policosanol comprises 1-tetracosanol, 1-hexacosanol, 1-octacosanol, 1-triacontanol, 1-dotriacontanol and 1-tetratriacontanol.

4. The composition of claim 3, wherein said policosanol has the following quantitative composition:
1-docosanol (C-22) 0-5wt %
1-tetracosanol (C-24) 0-30 wt %
1-hexacosanol (C-26) 5-30 wt %
1-heptacosanol (C-27) 5-10 wt %
1-octacosanol (C-28) 10-20 wt %
1-nonacosanol (C-29) 0-5 wt %
1-triacontanol (C-30) 5-40 wt %
1-dotriacontanol (C-32) 1-25 wt %
1-tetratriacontanol (C-34) 0 7 wt %.

5. The method of claim 1, wherein said inflammatory condition is selected from the group consisting of muscle inflammation, end-stage renal disease, diabetes, cardiovascular disease, metabolic syndrome, and respiratory inflammatory conditions.

6. The method of claim 1, wherein said composition is administered by parenteral, transdermal, intranasal, sublingual, transmucosal, intra-arterial, or intradermal administration.

7. The method of claim 1, wherein said composition is delivered to said subject as a controlled release composition.

8. The method of claim 7, wherein said controlled release composition comprises a flowable thermoplastic polymer composition comprising a biocompatible polymer, a biocompatible solvent, and policosanol, and is delivered to a bodily tissue or fluid in said subject, wherein the amounts of the polymer and the solvent are effective to form a biodegradable polymer matrix containing policosanol in situ when said composition contacts said bodily fluid tissue or fluid.

9. The method of claim 8, wherein said polymer is a poly(alkylene glycol) or a polysaccharide.

10. The method of claim 7, wherein the composition further comprises a controlled release additive.

11. The method of claim 8, wherein said biocompatible polymer is selected from the group consisting of polylactides, polyglycolides, polyanhydrides, polyorthoesters, polycaprolactones, polyamides, polyurethanes, polyesteramides, polydioxanones, polyacetals, polyketals, polycarbonates, polyorthocarbonates, polyphosphazenes, polyhydroxybutyrates, polyhydroxyvalerates, polyalkylene oxalates, polyacrylates, polyalkylene succinates, poly(malic acid), poly(amino acids) and copolymers, terpolymers, cellulose diacetate, ethylene vinyl alcohol, and copolymers and combinations thereof.

12. The method of claim 8, wherein the polymer matrix releases said policosanol by diffusion, erosion, or a combination of diffusion or erosion as the polymer matrix biodegrades in said subject.

13. The method of claim 8, wherein said policosanol is added to said polymer composition prior to administration such that said solid polymer matrix further contains said policosanol.

14. The method of claim 7, wherein said controlled release composition is in film form.

15. The method of claim 14, wherein said film comprises polylactic acid, polyglycolic acid and mixtures and copolymers thereof.

16. The method of claim 7, wherein said controlled release composition is in tablet form.

17. The method of claim 1, further comprising administering at least one additional anti-inflammatory agent, wherein said policosanol and said additional anti-inflammatory agent are administered as an admixture, separately and simultaneously, or separately in any order.

18. A method of reducing the level of C-reactive protein (CRP) in a subject, comprising administering to the subject an effective amount of a composition comprising policosanol.

19. The method of claim 18, wherein said policosanol comprises at least one higher primary aliphatic alcohol selected from straight chain primary aliphatic alcohols having 20 to 36 carbon atoms.

20. The composition of claim 19 wherein said policosanol comprises 1-tetracosanol, 1-hexacosanol, 1-octacosanol, 1-triacontanol, 1-dotriacontanol and 1-tetratriacontanol.

20. A kit comprising a first container comprising a controlled release formulation of policosanol, said formulation comprising an amount of policosanol effective to treat or reduce and/or prevent macular degeneration-related disorders.

21. The kit of claim 20, further comprising a puncture needle or catheter.
